(19) United States
(12) Patent Application Publication
(10) Pub. No.: US 2023/0279507 A1
(43) Pub. Date: Sep. 7, 2023

(54) METHOD FOR TESTING OF SAMPLE DAY MILK FOR PATHOGENS

(71) Applicant: Acumen Detection, LLC, Syracuse, NY (US)

(72) Inventors: Meghan F. Carter, Bridgeport, NY (US); Roger Saltman, Cazenovia, NY (US); Charles D. Stormon, Cazenovia, NY (US)

(73) Assignee: Acumen Detection, LLC, Syracuse, NY (US)

(21) Appl. No.: 18/116,894

(22) Filed: Mar. 3, 2023

Related U.S. Application Data
(60) Provisional application No. 63/316,537, filed on Mar. 4, 2022.

(51) Int. Cl. C12Q 1/689; C12Q 1/6895

(52) U.S. Cl. CPC C12Q 1/689; C12Q 1/6895

(57) ABSTRACT

A method of testing milk samples for the presence of pathogens. Sample day milk may be processed using specific reagents for the detection of pathogens and then subjected to polymerase chain reaction (PCR) amplification using multi-well plates for fast and accurate detection of the presence of nucleotides from specific pathogens. The sample day milk can be collected non-aseptically without compromising the accuracy of the detection results.

### BACKGROUND OF THE INVENTION

### Cross Reference to Related Application(s)

The present application relates and claims priority to U.S. Provisional Application No. 63/316,537, filed Mar. 4, 2022, the entire contents/disclosure of which is hereby incorporated by reference.

### 1. Field of the Invention

The present invention relates to milk testing methods and, more specifically, to a method for testing milk for pathogens.

### 2. Description of the Related Art

Dairy operations frequently test milk for a wide variety of reasons and applications. Milk testing requires accurate sample preparation and testing to prevent inconclusive results, misidentification and/or misdiagnosis issues, and other problems, all of which are expensive and time-consuming. Automating milk sample preparation and testing can prevent or mitigate against these expensive and time-consuming issues. However, these approaches usually involve specialized aseptic collection to avoid pathogens that may be present on the udder, teat, or a liquid on outside of teat but not in the milk. As a result, sample of milk obtained from normal dairy herd information (DHI) type collection processes cannot be used for such testing. Additionally, traditional milk sample analysis for pathogens can take twenty-four hours or longer to receive a diagnosis or other test results, a delay which is both time-consuming and expensive. Conventional collection processes. Accordingly, there is a need in the art for a testing approach that can process large numbers of samples of milk and that will provide accurate results even if the milk is collected using standard, non-aseptic collection processes.

### BRIEF SUMMARY OF THE INVENTION

The present invention provides an approach for milk testing that can be performed quickly and with high throughput. More specifically, the present invention includes the steps of collecting sample day milk and immediately (if desired) analyzing the samples in a multi-well system using specific reagents and polymerase chain reaction (PCR) amplification approaches. The present invention can be used on samples collected through normal dairy herd information (DHI) type collection process (i.e., not aseptic) as any pathogens present in the milk will be detected without false positives due to pathogens that might have been present on the udder, teat, or liquid on outside of teat. In addition, although the DHI collection has a preservative in it that kills any pathogens, the present invention remains capable of detecting the presence of such pathogen based on the presence of any remaining fragments of DNA.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a flowchart of a method of detecting pathogens in collection day milk samples according to the present invention.

FIG. 2 is a first graph of the results of a STREP assay according to the present invention.

FIG. 3 is a second graph of the results of a STREP assay according to the present invention.

FIG. 4 is a chart of the results for DHIA Complete-16 for the 10 highest SCC Samples in Comparison to Acu-Reagents STREP and MYPRO.

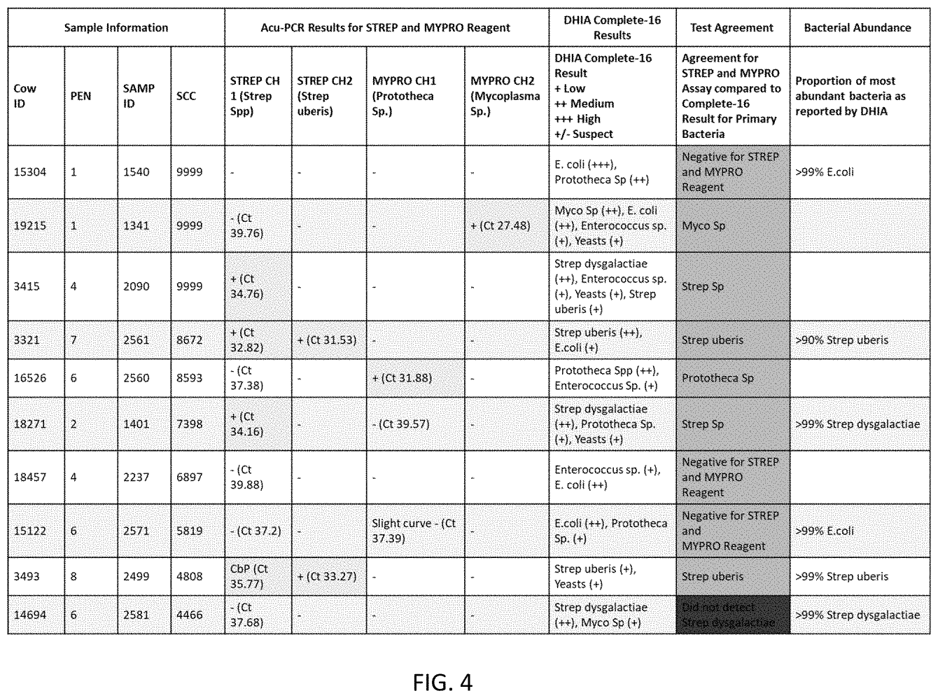

### DETAILED DESCRIPTION OF THE INVENTION

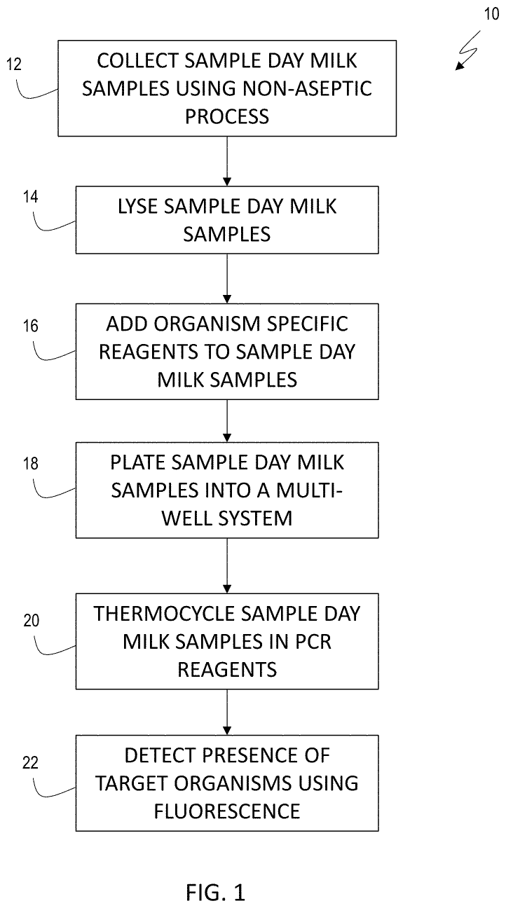

Referring to the figures, wherein like numerals refer to like parts throughout, there is seen in FIG. 1 a method 10 according to the present invention for milk testing that involves collecting sample day samples of milk 12, such as by conventional dairy herd information (DHI) type collection non-aseptic processes. Next, the samples are lysed 14 and organism specific reagents for the detection of any genetic material, such as DNA, or pathogenic organisms 16 are added. For example, oligonucleotides probes along with primers and fluorophores may be used. The samples are then plated in a multi-well system 18 and thermocycled for PCR amplification in the presence of standard PCR reagents 20. The presence of any target organisms may then be determined based on changes in fluorescence 22. Method 10 thus employs polymerase chain reaction (PCR) amplification using specific reagents according to the present invention to determine the presence of any deoxyribonucleic acid (DNA) indicating the presence of organisms associated with dairy herd infections. Analysis using method 10 is reliable even if the samples were collected using dairy herd information (DHI) type collection process that are non-aseptic and include the antimicrobials, such as bronopol, as method 10 is robust enough to identify the presence of contaminating organisms even after treatment with antimicrobials.

In a first study, samples were selected from cows whose PSCC<200,000 and SCC>400,000 in order to sort the new infections from the chronic infections. This sorting yielded 103 samples. Of the 103 samples, 51 cows had not been previously sampled but currently had a SCC>400,000 on the current test. 103 samples with requisite negative controls and positive controls (known bacterial concentrations of 10², 10³, 10⁴, and 10⁵ genome copies) seemed appropriate for this pilot. After the samples were sorted based on SCC criteria, the sample cups were wiped down with 70% IPA and processed through the Acu-Lysis Reagent. Once processing was completed, the 103 processed samples were placed into the 3.5° C. refrigerator for overnight storage. The samples were then tested to determine if DNA was present from Staph aureus, Coagulase Negative Staphs, and/or Mycoplasma bovis. Of the 103 samples, the following were found: 0 Staph aureus; 2 Coagulase Negative Staphs; 0 Mycoplasma bovis.

Following the completion of the STAPH and MYCOB runs, the STREP reagent was tested to determine if it might be useful for these samples. A subset of 10 samples were selected based on the sample being among the 10 highest SCC (383,800<SCC<1,000,000). 3/10 of the samples gave strong positives for Strep uberis and Strep species. 3/10 of the samples demonstrated late amplification and were considered close but negative (Retest). 4/10 of the samples demonstrated no amplification and are negative. Table 1 below shows the results:

TABLE 1

| Cow ID | SCC | S. uberis Result | Strep Spp. Result |
|---|---|---|---|
| 3207 | 9823 | Retest | Retest |
| 16890 | 9999 | − | − |
| 16250 | 5444 | Retest | Retest |
| 16161 | 4642 | + | + |
| 19167 | 5713 | Retest | Retest |
| 17111 | 3839 | + | + |
| 14813 | 9875 | + | + |
| 18885 | 5608 | − | − |
| 18798 | 3857 | − | − |
| 3680 | 8156 | − | − |

Table 2 below shows a summary of all results from the sample day.

TABLE 2

| STAPH CH1 | STAPH CH2 | MYCOB CH2 | STREP CH1 | STREP CH2 |
|---|---|---|---|---|
| (S. aureus) | (Staph Spp) | (M. bovis) | (Strep Spp) | (Strep uberis) |
| Total | Total | Total | Total | Total |
| Positive: 0 | Positive: 0 | Positive: 0 | Positive: 3 | Positive: 3 |
| Total | Total | Total | Total | Total |
| Retest: 0 | Retest: 0 | Retest: 0 | Retest: 3 | Retest: 3 |
| Total | Total | Total | Total | Total |
| Negative: | Negative: | Negative: | Negative: 4 | Negative: 4 |
| 103 | 103 | 103 | | |
| Total | Total | Total | Total | Total |
| Samples: | Samples: | Samples: | Samples: 10 | Samples: 10 |
| 103 | 103 | 103 | | |

The results show that sample day milk can be used for analysis with Acu-PCR. Contaminated samples have very late amplification with levels of DNA present that are well below the Level of Quantification. These results also show that milk samples can be analyzed in a 96 well system to quickly (<3 hrs.) get Acu-PCR results. Test day samples with bronopol can be used in a 96 well plate with appropriate positive and negative controls in the Acu-PCR system.

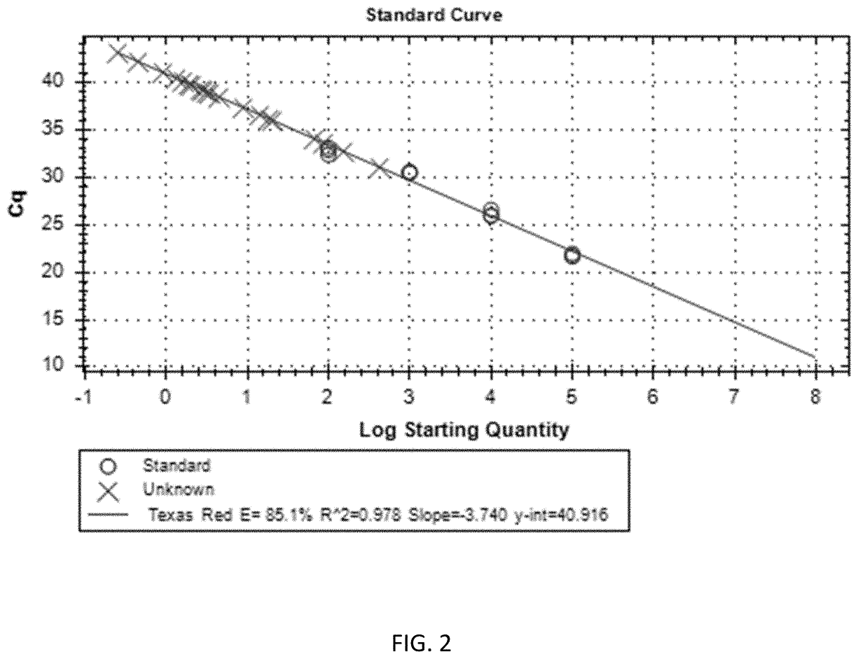

In a second study, the cow selection criteria were changed to include cows with HIGHEST Test Day SCC (down to 400,000)−218 cows. PCR tests included the STREP Reagent (Strep uberis and Strep spp.) as well as the STAPH Reagent (Staph aureus and Staph spp.) and 1:4 Dilutions of milk were evaluated to see if the approach was still sensitive enough to detect DNA. FIGS. 1 and 2 show the results of the second study, with Table 3 below showing the STREP and STAPH Results for all 218 Feb. Samples.

TABLE 3

| STREP CH1 | STREP CH2 | STAPH CH1 | STAPH CH2 |
|---|---|---|---|
| (Strep spp) | (Strep uberis) | (S. aureus) | (Staph spp) |
| Total | Total | Total | Total |
| Positive: 10 | Positive: 10 | Positive: 1 | Positive: 0 |
| Total Indeter- | Total Indeter- | Total Indeter- | Total Indeter- |
| minate: 7 | minate: 6 | minate: 3 | minate: 0 |
| Total | Total | Total | Total |
| Negative: 201 | Negative: 202 | Negative: 214 | Negative: 218 |
| Total | Total | Total | Total |
| Samples: 218 | Samples: 218 | Samples: 218 | Samples: 218 |

Table 4 below shows the Pooling Results with the STREP Reagent.

TABLE 4

| | | | STREP CH1 | STREP CH2 |
|---|---|---|---|---|
| | | | Strep spp | Strep uberis |
| | | | (Positive | (Positive |
| Cow ID | Sample | Type | below Ct 35.5) | below Ct 35.5) |
| 15215 | 1334 | Negative | −(Ct 42.96) | −(Ct N/A) |
| 3493 | 2499 | Positive Sample | +(Ct 34.41) | +(Ct 29.39) |
| | | (Strep Spp, | | |
| | | Strep uberis) | | |
| 18136 | 2529 | Positive Sample | +(Ct 35.31) | +(Ct 31.74) |
| | | (Strep Spp, | | |
| | | Strep uberis) | | |
| 16185 | 2566 | Positive Sample | +(Ct 31.72) | +(Ct 27.95) |
| | | (Strep Spp, | | |
| | | Strep uberis) | | |
| 3321 | 2561 | Positive Sample | +(Ct 31.67) | +(Ct 28.21) |
| | | (Strep Spp, | | |
| | | Strep uberis) | | |
| 15215 | Pool 1 | (2499 + 1334) | Retest | +(Ct 33.63) |
| 3493 | | 1:4 Ratio | (Ct 36.82) | |
| 18136 | Pool 2 | (2529 + 1334) | +(Ct 35.25) | +(Ct 35.42) |
| 15215 | | 1:4 Ratio | | |
| 16185 | Pool 3 | (2566 + 1334) | Retest | +(Ct 30.33) |
| 15215 | | 1:4 Ratio | (Ct 35.88) | |
| 3321 | Pool 4 | (2561 + 1334) | +(Ct 32.54) | +(Ct 32.2) |
| 15215 | | 1:4 Ratio | | |

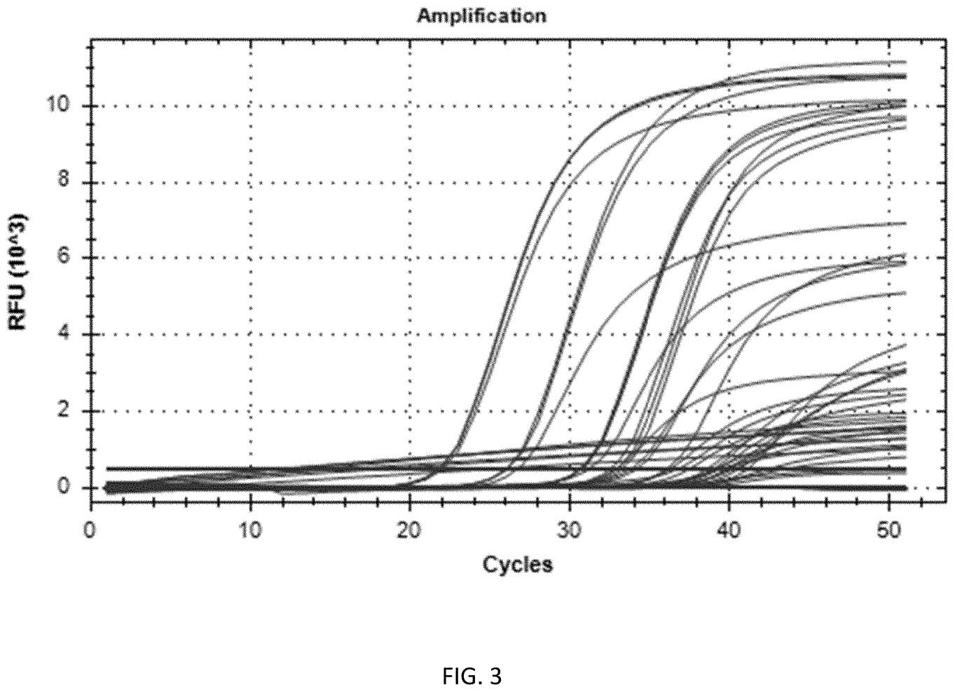

FIG. 3 shows the Results for DHIA Complete-16 for the 10 highest SCC Samples in Comparison to Acu-Reagents STREP and MYPRO. The use of Acumen Reagents in a 96 well Bio-Rad plate can provide a valuable screening test using DHI Samples. Bronopol does not affect the results. Low level contamination in DHI Samples does not affect results. In fact, 1:4 Dilutions of milk were still sensitive enough to detect DNA (dependent on amount of DNA coming from target organisms).

Knowledge of prevalent mastitis-causing organisms and prevalence level of pathogens in the herd can help with the reagent selection/pooling decision. Prevalence of >10% or unknown warrants testing each cow in the group vs. pooling. If problem pathogens are known, it is possible to focus on the use of appropriate Acu-Reagents. If problem pathogens are unknown, initial testing with MYPRO and SASUB is indicated.

Materials and Methods

Sample Storage

Non-aseptic milk samples preserved in 16% Bronopol and obtained during regular Dairy Herd Improvement Association collection. Sample Days are received and stored at 3.5° Celsius. The samples are then sorted based on previously determined Somatic Cell Count thresholds to increase the likelihood that they would yield a positive result.

Sample Processing with Acu-Lysis Reagent

Once sorted, the samples are then placed into a Biosafety Cabinet. Each sample is mixed using a vortex and 1 mL of preserved raw milk and is transferred to the Acu-Lysis Reagent Tube A. This Tube A is then inverted to mix and placed back into the tube rack until all the samples had been transferred to their respective Tube As and inverted to mix. Following the mixing steps, all Tube As are placed into dry heat baths at a temperature of 100° C. for 20 minutes. Following the conclusion of the 20-minute heat step, the samples are removed from the heat block and allowed to cool for one or more minutes. Following the cooling period, the Tube As are placed into a centrifuge and spun for approximately 10 seconds. This step is repeated until all the Tube As had been centrifuged. The Tube As are then placed into the Biosafety Cabinet and 200 uL from each tube A is transferred to a corresponding Acu-Lysis Reagent Tube B. Following the transfer, Tube B is inverted to mix. At this point the sample processing is complete and the Tube Bs are placed into the 3.5° Celsius fridge and refrigerated overnight.

PCR Reagent Loading Procedure Unknown Samples

Single use lyophilized or premixed Acu-Reagent contained in a 0.2 mL high profile optically clear PCR tube are loaded with 100 uL of the Tube B solution from each sample. The Tube is then flicked to mix and placed into a PCR Tube Rack. Either one of the following two options may then be used.

First, following the loading of 12 consecutive PCR Tubes and aligning them in a single row of the PCR Tube Rack, the caps are opened for each individual PCR tube and a 12-channel pipette is used to transfer approximately 100 uL from each PCR Assay Tube to the corresponding well in a 96-Well Hardshell BioRad Plate, assigned by random number generation in an Excel Spreadsheet.

Second, the pre-populated liquid or lyophilized Acu-Reagent 96-well plate is loaded with 100 uL of sample into each well as assigned by random number generation in an Excel Spreadsheet and pipetted to mix.

The Acu-Reagents suitable for use with the method described above are the MYPRO Reagent, the MYCOB Reagent, the SASUB Reagent, the STAPH Reagent, and the STREP Reagent. The Acu-Reagents are all ready-to-use lyophilized one channel (simplex) or two channel (duplex) PCR reagents packaged in 0.2 ml plastic tubes with 10 microliters of PCR master mix, primers, probes (oligonucleotides), fluorophores for in vitro detection of target DNA sequences in the associated organisms. For example, MYPRO is a duplex reagent that detects oligonucleotides from Mycoplasma species and Prototheca species. MYCOB is a simplex reagent that detects oligonucleotides from Mycoplasma bovis, SASUB is a duplex reagent that detects oligonucleotides from Staph aureus and Strep uberis, STAPH is a duplex reagent that detects oligonucleotides from Staph species and Staph aureus, STREP is a duplex reagent that detects oligonucleotides from Strep species and Strep uberis. It should be recognized by those of skill in the art that other reagents may be used including those to detect oligonucleotides from specific organisms as desired.

PCR Reagent Loading Procedure Positive Controls/Standards

Single use lyophilized or premixed Acu-Reagents contained in a 0.2 mL high profile optically clear PCR tube are loaded with 99 uL of molecular grade Hyclone Water. The tube is then flicked to mix and placed into a PCR tube rack. 1 uL of genomic DNA is then added to each positive control with triplicates of each of the following concentrations: 10⁵ Genome Copies/uL, 10⁴ Genome Copies/uL, 10³ Genome Copies/uL, and 10² Genome Copies/uL. For example, the STAPH Reagent would be loaded with Staph aureus gDNA purchased from ATCC and used as the standard genomic material. For another example, the STREP Reagent would be loaded with Strep uberis gDNA purchased from ATCC and used as the standard genomic material. Either of the two following approaches may then be used.

First methodology—Following the loading of 12 consecutive PCR tubes and aligning them in a single row of the PCR Tube Rack, the caps are opened for each individual PCR tube and a 12-channel pipette is used to transfer approximately 100 uL from each PCR assay tube to the corresponding well in a 96-Well Hardshell BioRad Plate, as assigned by a random number generator in an Excel spreadsheet.

Second methodology—The pre-populated liquid or lyophilized Acu-Reagent 96-well plate is loaded with 99 uL of molecular grade Hyclone Water into each well and pipetted to mix. 1 uL of genomic DNA is then added to each positive control, with triplicates of each of the following concentrations: 10⁵ Genome Copies/uL, 10⁴ Genome Copies/uL, 10³ Genome Copies/uL, and 10² Genome Copies/uL. For example, the STAPH Reagent, would be loaded with Staph aureus gDNA purchased from ATCC and used as the standard genomic material. Another example, the STREP Reagent, would be loaded with Strep uberis gDNA purchased from ATCC and used as the standard genomic material.

The Acu-Reagents suitable for use with the method described above are the MYPRO Reagent, the MYCOB Reagent, the SASUB Reagent, the STAPH Reagent, and the STREP Reagent. The Acu-Reagents are all ready-to-use lyophilized one channel (simplex) or two channel (duplex) PCR reagents packaged in 0.2 ml plastic tubes with 10 microliters of PCR master mix, primers, probes (oligonucleotides), fluorophores for in vitro detection of target DNA sequences in the associated organisms. For example, MYPRO is a duplex reagent that detects oligonucleotides from Mycoplasma species and Prototheca species. MYCOB is a simplex reagent that detects oligonucleotides from Mycoplasma bovis, SASUB is a duplex reagent that detects oligonucleotides from Staph aureus and Strep uberis, STAPH is a duplex reagent that detects oligonucleotides from Staph species and Staph aureus, STREP is a duplex reagent that detects oligonucleotides from Strep species and Strep uberis. It should be recognized by those of skill in the art that other reagents may be used including those to detect oligonucleotides from specific organisms as desired. It should be recognized by those of skill in the art that other reagents may be used including those to detect oligonucleotides from specific organisms as desired.

Thermocycler Setup: Once all samples are loaded into the 96-Well Plate(s), the plate is sealed using a BioRad Microseal® ‘C.’ PCR Plate Sealing Film. The plate is then placed into a BIORAD CFX96 C1000 Thermocycler and a run is made using User-Defined Parameters. The cycling protocol selected is the previously established proprietary cycling temperatures and cycle numbers. The plate file is created using the BioRad Manager Version 3.1. This plate is set to detect fluorescent amplification for the FAM Channel and Texas Red Channel which corresponds to the fluorescent emission generated by the STAPH and STREP Reagents. Each sample name is then added to the plate using the Spreadsheet/Excel Importer function of the BioRad Software. The samples identified as standards (gDNA Positives) are assigned as such in the BioRad software as well as assigned the log starting concentration of genome copies and selected as replicates. Negative controls are also identified as such using the BioRad Software with all other samples identified as “Unknown.” The plate file is then saved for future use in experiments with a similar setup. The “Run” button is then clicked and the thermocycler begins the heating and cooling protocol as well as simultaneously recording amplification/fluorescence for the identified channels.

Result Interpretation: Following the completion of the run, the BioRad software populates a standard curve with the known positives and the unknowns plotted against log concentration and Cq values. The comparison of the unknowns to the standard curve generated allows us to set a Cq threshold of 35.5 to separate positive samples (below 35.5) from those that had environmental, and carry-over contaminants (above 35.5).

Significance: This ability to separate contaminants from positives with DHI Samples is a novel approach as it eliminates the time-consuming effort of obtaining individual aseptic samples from individual cows and expedites the results turnaround time when paired with the Acu-Reagents.

What is claimed is:

1. A method of detecting a target pathogen in a plurality of cows in a herd, comprising the steps of:
collecting a plurality of samples of milk from said herd, wherein each of said plurality of samples of milk is collected from a different one of said plurality of cows;
exposing each of said plurality of samples of milk to a lysing agent;
adding at least one reagent each of said plurality of samples of milk, wherein said at least one reagent is configured to identify any genetic material associated with at least one pathogen that may be present in said herd;
amplifying any genetic material in said plurality of samples of milk using polymerase chain reaction; and
detecting any change in fluorescence of each of said plurality of samples of milk after amplification to determine whether each of said plurality of samples of milk contains genetic material of said at least one pathogen.

2. The method of claim 1, wherein said step of collecting said plurality of samples of milk from said herd comprises a non-aseptic collection.

3. The method of claim 2, wherein said non-aseptic collection comprises a dairy herd information (DHI) collection.

4. The method of claim 1, wherein said at least one reagent comprises a probe configured to identify an organism from the genus Mycoplasma.

5. The method of claim 4, wherein said probe is configured to identify Mycoplasma bovis.

6. The method of claim 1, wherein said at least one reagent comprises a probe configured to identify an organism from the genus Prototheca.

7. The method of claim 1, wherein said at least one reagent comprises a probe configured to identify an organism from the genus Staphylococcus.

8. The method of claim 7, wherein said probe is configured to identify Staphylococcus aureus.

9. The method of claim 1, wherein said at least one reagent comprises a probe configured to identify an organism from the genus Streptococcus.

10. The method of claim 9, wherein said probe is configured to identify Streptococcus uberis.

11. The method of claim 1, wherein said at least one reagent comprises a duplex reagent for identifying a Mycoplasma species and a Prototheca species.

12. The method of claim 1, wherein said at least one reagent comprises a duplex reagent for detecting a Staph species and Staph aureus.

13. The method of claim 1, wherein said at least one reagent comprises a duplex reagent for detecting a Strep species and Strep uberis.

14. The method of claim 1, wherein said step of amplifying any genetic material in said plurality of samples of milk using polymerase chain reaction and said step of detecting any change in fluorescence of each of said plurality of samples of milk after amplification are performed on a real-time polymerase chain reaction (PCR) thermocycler.
